(19) United States
(12) Patent Application Publication
(10) Pub. No.: US 2026/0026954 A1
(43) Pub. Date: Jan. 29, 2026

(54) SOFT KNEE BRACE

(71) Applicant: DJO LLC, Carlsbad, CA (US)

(72) Inventors: Luc THYS, Kortenberg (BE); Julien OXOTEGUY, Cambo (FR)

(21) Appl. No.: 19/343,696

(22) Filed: Sep. 29, 2025

Related U.S. Application Data
(63) Continuation of application No. PCT/US2024/022322, filed on Mar. 29, 2024.
(60) Provisional application No. 63/500,400, filed on May 5, 2023.

(51) Int. Cl. A61F 5/01

(52) U.S. Cl. CPC A61F 5/0106

(57) ABSTRACT

A knee brace for relieving pressure on a portion of a knee includes a body with one or more supports arranged along the body. The one or more supports can be positioned on a medial section and lateral section of the body of the knee brace. The knee brace can have a first tightening mechanism and a second tightening mechanism which can be configured to incrementally tension the knee brace to apply biomechanical forces to a leg of the patient or wearer.

### TECHNICAL FIELD

The present disclosure relates to orthopedic braces. In particular, knee braces for relieving pressure on a portion of a knee is disclosed.

### BACKGROUND

Osteoarthritis, degenerative meniscus, knee instability, post traumatic unicompartmental cartilage damage and other knee ailments are conditions which can affect the natural cushioning of knee joints. In the United States, osteoarthritis is one of the most frequent causes of physical disability as a large portion of the population suffers from this condition.

Unicompartmental knee osteoarthritis can occur when one region or compartment of a knee is affected or injured, while other regions or compartments of the knee remain healthy or undamaged. Patients suffering from unicompartmental knee osteoarthritis may benefit a brace or system which can apply biomechanical forces to a leg of the patient to shift the biomechanical load from the affected compartment of the knee to a healthy portion of the knee.

### SUMMARY

Embodiments of devices and methods for relieving pressure on a portion of a knee are disclosed.

One innovation includes a knee brace having a body with a flexible textile substrate. The body can have a distal portion configured to surround a portion of a calf of a wearer and extend below a knee of the wearer. The distal portion can include a first end and a second end. The first end and the second end can be coupled together with a repeatable pattern. The body can also have a proximal portion configured to surround a portion of a thigh of the wearer and extend above the knee of the wearer. In some embodiments, the flexible textile substrate can be made of a soft and/or elastomeric material. Additionally, the flexible textile substrate can be most elastic when tension is applied in a lateral direction. The body can have an outward facing surface and an inward facing surface on the proximal portion and the distal portion. The inward facing can be configured to face an opposite direction of the outward facing surface. In some embodiments, a fastener can be configured to couple the outward facing surface of the proximal portion and the inward facing surface of the proximal portion. The fastener can be a hook and loop fastener. In some embodiments, an anti-slip portion can be located on the inward facing surface of the proximal portion. Additionally, one or more supports can be positioned on the body. The one or more supports can be semi-rigid. The one or more supports can include a first support positioned laterally on the outward facing surface of the distal portion of the body, a second support positioned laterally on the outward facing surface of the proximal portion of the body, a third support positioned medially on the outward facing surface of the distal portion of the body, and a fourth support positioned medially on the outward facing surface of the proximal portion of the body. Furthermore, in some embodiments, a first tightening mechanism can be positioned on the knee brace. The first tightening mechanism can include a first rotating dial and a first cable. The first rotating dial can be positioned on the first support. The first cable can be coupled to the first support and the third support. A second tightening mechanism can be positioned on the knee brace and can include a second rotating dial and a second cable. The second rotating dial can be positioned on the second support. The second cable can be coupled to the second support and the fourth support. The knee brace can have a hinge positioned laterally on the knee brace. The hinge can be coupled to the first support and the second support. Additionally, the distal portion of the body and the proximal portion of the body can be configured to encircle the knee of the wearer. The first tightening mechanism can be configured to incrementally tension the distal portion by rotating the first rotating dial which can increase the tension in the first cable. The first tightening mechanism can also be configured to release tension in the distal portion by releasing the tension in the first cable. Additionally, the second tightening mechanism can be configured to incrementally tension the proximal portion by rotating the second rotating dial to increase the tension in the second cable. The second tightening mechanism can be configured to release tension in the proximal portion by releasing the tension in the second cable.

In some embodiments disclosed herein, the one or more supports can include one or more additional features, or different features. The first support can include an opening sized and configured for the first rotating dial. Further, in some embodiments, the second support can include an opening sized and configured for the second rotating dial. The fourth support can include a first component and a second component, wherein the first component is removably coupled to the second component. The second component can include a pulling aid which can be coupled to the second component, and which can provide increased tension to the proximal portion. In some embodiments, the one or more supports can be coupled to the body with a repeatable pattern which can be a zig-sag sewing pattern.

In some embodiments disclosed herein, the first tightening mechanism and second tightening mechanisms can include one or more cable guides. The one or more cable guides can have one or more sided closures stitched into the one or more cable guides to retain the first or second cable. Additionally, the one or more cable guides can include one or more hidden closures stitched into the one or more cable guides to retain the first or second cable. Further, in some embodiments, the one or more cable guides can include one or more clamps, the one or more clamps are removably mounted (e.g., removably fastened) onto the one or more cable guides to retain the first or second cable. The one or more cable guides can be fastened to the body with a zig-zag sewing pattern.

In some embodiments, the one or more tightening mechanisms can include one or more additional features, or different features. The first cable of the first tightening mechanism can extend from a first location on the first support to a first location on the third support. Furthermore, in some embodiments, the first cable can extend to a second location on the third support and then to a second location on the first support. Continuing, the first cable can extend to a third location on the third support to a fourth location on the third support, to a third location of the first support to the first location at the first support. The first cable can be between 80 cm and 110 cm. The second cable of the second tightening mechanism can extend from a first location on the second support to a first location on the fourth support. Furthermore, the second cable can extend to a second location on the fourth support to a second location of the second support. Continuing, the second cable can extend to a third location on the fourth support to a fourth location on the fourth support. The second cable can then extend to a third location on the second support to the first location on the second support. The second cable can have a length between 90 cm and 190 cm.

In some embodiments disclosed herein, the hinge can include one or more additional features, or different features. The hinge can be located on a lateral side of the knee of the wearer and be configured to support movement of the knee of the wearer. The hinge can include a first assembly bar and a second assembly bar. The first assembly bar and second assembly bar can have a distal end and a proximal end. The proximal ends of the first and second assembly bars can be coupled to the hinge. The distal ends of the first and second assembly bars can extend into a portion of the first and second supports, respectively. The first and second assembly bars can engage with slots positioned on the first and second supports, respectively. In some embodiments, the knee brace can include a condyle pad which can be located between the hinge and a condyle area of the knee of a wearer. The condyle pad can be a compliant material. The condyle pad can be configured to control an amount of pressure applied by the hinge to the condyle area of the knee of a wearer. Additionally, in some embodiments, the condyle pad can have a thickness of between about 5 mm and 18 mm.

In some embodiments disclosed herein, the knee brace can include one or more additional features. The knee brace can include a lateral flexible stay which can be positioned on a medial portion of the body. The lateral flexible stay can be made of flexible steel. Additionally, in some embodiments, the lateral flexible stay can be positioned on a lateral portion of the body. The lateral flexible stay can include a first end which extends to a position connectable to the third support and a second end which extends to a position connectable to the fourth support. The body of the knee brace can include a cover which can be coupled to the lateral flexible stay. The cover can be positioned on the inward facing surface of the body. The cover can extend from the third support to the fourth support. Additionally, the cover can extend from the first support to the second support. Additionally, the lateral flexible stay can have a length between approximately 200 mm and approximately 240 mm.

In some aspects, the techniques described herein can relate to a method for providing stability to a knee joint, which can include a knee brace. The method can include positioning the distal portion of the flexible textile substrate of the knee brace around the calf of the wearer and below the knee of the wearer. The method can also include positioning the proximal portion of the flexible textile substrate around the thigh of the wearer and above the knee of the wearer. Further, the method can include positioning the proximal portion of the flexible textile substrate and the distal portion of the flexible textile substrate around the knee of the wearer. The method can include positioning the first support and the second support on a lateral side of the wearer. The method can include positioning the third support and the fourth support on a medial side of the wearer. The method can include tightening the first tightening mechanism to incrementally tension the distal portion of the flexible textile substrate by rotating the first rotating dial to increase tension in the first cable. Additionally, the method can include tightening the second tightening mechanism to incrementally tension the proximal portion by rotating the second rotating dial to increase tension in the second cable. The method can include loosening the first tightening mechanism to release tension in the distal portion by releasing tension in the first cable and loosening the second tightening mechanism to release tension in the proximal portion by releasing tension in the second cable. In some embodiments of the method the hinge assembly can be positioned on a lateral side of the wearer. The hinge assembly can allow the knee to flex and extend on a sagittal plane.

In some embodiments, the method can include tightening the first tightening mechanism to incrementally tension the distal portion when the first cable extends from a first location on the first support to a first location on the third support, to a second location on the third support to a second location on the first support, to a third location on the third support to a fourth location on the third support, to a third location of the first support to the first location at the first support. In some embodiments, the method can include tightening the second tightening mechanism to incrementally tension the proximal portion when the second cable extends from a first location on the second support to a first location on the fourth support, to a second location on the fourth support to a second location of the second support, to a third location on the fourth support to a fourth location on the fourth support, to a third location on the second support to the first location on the second support.

### BRIEF DESCRIPTION OF THE DRAWINGS

The features and advantages of the embodiments described herein will become more fully apparent from the following description and appended claims, taken in conjunction with the accompanying drawings. These drawings may depict only certain embodiments in accordance with the disclosure and are not to be considered limiting of its scope. In the drawings, similar reference numbers or symbols typically identify similar components, unless context dictates otherwise. In some instances, the drawings may not be drawn to scale.

FIG. 1 illustrates an example of a front perspective view of a knee brace.

FIG. 2 illustrates an example of a lateral side perspective view of the knee brace of FIG. 1.

FIG. 3 illustrates an example of a medial side perspective view of the knee brace of FIG. 1.

FIG. 4 illustrates an example of a rear perspective view of the knee brace of FIG. 1.

FIG. 5 illustrates an example of an alternative perspective view of the knee brace of FIG. 1, showing a base layer of the knee brace including a closure system for the proximal portion of the base layer.

FIG. 6A illustrates an example of a component of the knee brace of FIG. 1 for fixedly closing a proximal portion of the knee brace and providing counterforces to a thigh area.

FIG. 6B illustrates an example of a second component of the knee brace of FIG. 1 for freely closing a proximal portion of the knee brace and providing counterforces to a thigh area.

FIG. 6C illustrates an example of a support that can be used with the knee brace of FIG. 1 which can apply a counterforce to a medial portion of the calf area.

FIG. 6D illustrates an example of another support that can be used with the knee brace of FIG. 1 which can apply cushioning to a lateral portion of the calf area.

FIG. 6E illustrates an example of another support that can be used with the knee brace of FIG. 1 which can apply cushioning to a lateral portion of the thigh area.

FIG. 6F illustrates an example of another support combining the first component of FIG. 6A with the second component of FIG. 6B.

FIG. 7 illustrates a side perspective view of a hinge on the knee brace of FIG. 1.

FIG. 8A illustrates a front perspective view of the path of cables for providing compression and controlling the unloading of the knee brace of FIG. 1.

FIG. 8B illustrates a side perspective view of the path of cables for providing compression and controlling the unloading of the knee brace of FIG. 1.

FIG. 8C illustrates an alternative side perspective view of the path of cables for providing compression and controlling the unloading of the knee brace of FIG. 1.

FIG. 9 illustrates a guide for the cables of the knee brace of FIG. 1.

FIG. 10 illustrates an alternative view of guides for cables of the knee brace of FIG. 1.

FIG. 11A illustrates one embodiment of a guide for cables of the knee brace of FIG. 1.

FIG. 11B illustrates another embodiment of a guide for cables of the knee brace of FIG. 1.

FIG. 11C illustrates another embodiment of a guides for cables of the knee brace of FIG. 1.

FIG. 12A illustrates another embodiment of a guide for cables of the knee brace of FIG. 1.

FIG. 12B illustrates another embodiment of a guide for cables of the knee brace of FIG. 1.

FIG. 12C illustrates another embodiment of a guide for cables of the knee brace of FIG. 1.

FIG. 12D illustrates another embodiment of a guide for cables of the knee brace of FIG. 1.

FIG. 13A illustrates a rear perspective view of paths for guides for cables of the knee brace of FIG. 1.

FIG. 13B illustrates a front perspective view of paths for guides for cables of the knee brace of FIG. 1.

FIG. 14A illustrates a view of the inner surface of the knee brace of FIG. 1.

FIG. 14B illustrates an alternative view of the inner surface of the knee brace of FIG. 1.

FIG. 15 illustrates an alternative view of the inner surface of the knee brace of FIG. 1.

### DETAILED DESCRIPTION

The following detailed description describes embodiments of knee braces and methods, some of which are illustrated in the figures. These embodiments are not intended to be limiting, and various modifications, variations, combinations, etc., of the features of these embodiments are possible and within the scope of this disclosure.

In some embodiments, the knee braces described herein provide stabilization, support, bracing, compression, and/or pain relief for the knee joint. The knee brace may be used to provide relief for mild to moderate unicompartmental knee osteoarthritis and other knee conditions that may benefit from unicompartmental unloading. Such conditions may include, but are not limited to post-traumatic unicompartmental cartilage damage, post-surgical therapy, general instability of the knee joint by age, and/or unicompartmental knee replacement. Additionally, in some embodiments, the operating principles of this knee brace may apply forces to a leg or leg joint which can shift load from an affected, injured, or unstable portion of a knee to a healthy or unaffected portion of a knee.

In some embodiments, the knee braces disclosed herein can have plurality of supports, each of which can be designed to be positioned on a portion of a wearer's leg to provide support. The components can apply support and rigidity to a user's leg by unloading the affected areas of the knee. Additionally, in some embodiments, the knee brace can have a hinge system which can support the knee of the wearer and can guide the wearer's leg along a fixed path to help provide more optimal loading of the knee joint.

In some embodiments, the knee braces disclosed herein are configured to be patient specific. For example, in some embodiments, the size of the knee braces and the supports can be configured to be sized based on the thigh circumference or calf circumference of the user. Additionally, the knee braces disclosed herein can include cabling systems which can provide support and/or tension on portions of the wearer's leg based on the needs of the particular wearer. In some embodiments, the cable length can be based on the size or particular requirements of the wearer. Furthermore, in some embodiments, the knee brace can be made of a flexible textile material. The flexible textile material can be a knitted sleeve or another elastomeric material which can conform to the leg of the wearer. Advantageously, the flexible textile material can be such a material that it is convenient for everyday wear since the material can be a breathable material. In some embodiments, due to the flexible textile material, the knee brace can be comfortable to place over the clothes of a wearer, even if the wearer is not in athletic clothing. The flexible textile material can, in some embodiments, provide compression and reinforce the support/bracing systems of the components to improve the joint function and pain relief of the wearer.

Embodiments of a knee brace are described herein. The following is a list of certain components that are described and enumerated in this disclosure in reference to the above-listed figures. Other components, or aspects of these components, may not be included in the list but are disclosed in the figures and description. Accordingly, any aspect illustrated in the figures, whether or not called out separately herein, can form a portion of various embodiments and may provide basis for claim limitation relating to such aspects, with or without additional description. The table below lists certain enumerated components:

| Figure No. | Figure Description |
|---|---|
| 100 | Knee brace |
| 102 | Body |
| 104 | Flexible textile substrate |
| 106 | Distal portion of the body |
| 107 | First end of the distal portion of the body |
| 108 | Second end of the distal portion of the body |
| 109 | Bottom edge of the distal portion of the body |
| 110 | Top edge of the distal portion of the body |
| 112 | Proximal portion of body |
| 113 | Top edge of the proximal portion of the body |
| 114 | Bottom edge of the proximal portion of the body |
| 115 | Anti-slip portion |
| 116 | Outward facing surface of the body |
| 118 | Inward facing surface of the body |
| 120A | Fastener |
| 120B | Fastener |
| 121A | Fastener edge |
| 121B | Fastener edge |
| 122 | One or more supports |
| 124 | First support |
| 125 | Opening on first support |
| 126 | First slot |
| 127 | Edge of first support |
| 128 | Second support |
| 129 | Opening on second support |
| 130 | Second slot |
| 131 | Edge of second support |
| 132 | Third support |
| 133 | Edge of third support |
| 134 | Fourth support |
| 135 | First component |
| 136 | Second component |
| 137 | pulling aid |
| 138 | Edge of fourth support |
| 139A, 139B | Connector opening of second component |
| 140 | Medial section of the knee brace |
| 141 | Medial distal section of the knee brace |
| 142 | Medial proximal section of the knee brace |
| 143 | Lateral section of the knee brace |
| 144 | Lateral distal section of the knee brace |
| 145 | Lateral proximal section of the knee brace |
| 146A, 146B | Connector on the first component |
| 147 | One or more tightening mechanisms |
| 148 | First tightening mechanism |
| 150 | First rotating dial |
| 152 | First cable |
| 154 | Second tightening mechanism |
| 156 | Second rotating dial |
| 158 | Second cable |
| 160 | Hinge |
| 161 | Condyle pad |
| 162 | First assembly bar |
| 163 | Distal end of the first assembly bar |
| 164 | Proximal end of the first assembly bar |
| 166 | Second assembly bar |
| 167 | Distal end of the second assembly bar |
| 168 | Proximal end of the second assembly bar |
| 170 | Cable guide |
| 171 | Stitching for cable guide |
| 172 | Sided closure |
| 173 | Hidden closure |
| 174 | Clamps |
| 175 | Position of cable guide |
| 175A1 | Upper first position |
| 175A2 | Lower first position |
| 175B1 | Upper second position |
| 175B2 | Lower second position |
| 175C1 | Upper third position |
| 175C2 | Lower third position |
| 175D1 | Upper fourth position |
| 175D2 | Lower fourth position |
| 176 | First location on first support |
| 177 | First location on third support |
| 178 | Second location on third support |
| 179 | Second location on first support |
| 180 | Third location on third support |
| 181 | Fourth location third support |
| 182 | Third location on first support |
| 184 | First location on second support |
| 185 | First location on fourth support |
| 186 | Second location on fourth support |
| 187 | Second location on second support |
| 188 | Third location on fourth support |
| 189 | Fourth location on fourth support |
| 190 | Third location on second support |
| 192 | Lateral flexible stay |
| 193 | First end of the lateral flexible stay |
| 194 | Second end of the lateral flexible stay |
| 195 | Cover |
| K | Knee |

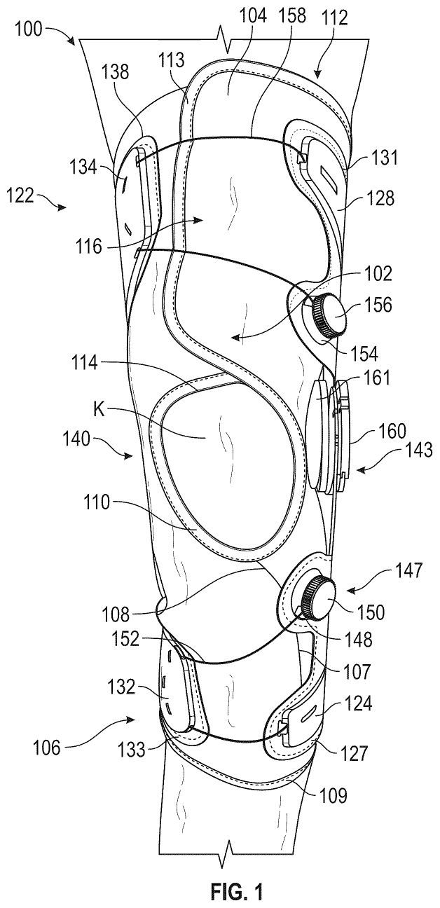

With reference to FIG. 1, an example of a knee brace 100 is illustrated. FIG. 1 shows a front perspective view of a knee brace 100 on a wearer's left leg. The knee brace 100 includes a body 102, where the body 102 can be made of, or include, a flexible textile substrate 104. The flexible textile substrate 104 can be made of a soft material and/or an elastomeric material. The flexible textile substrate 104 can be most elastic in a lateral direction, which can allow the knee brace 100 to stretch to surround the leg of the wearer. As used herein, “flexible” generally refers to the ability (e.g., of a material or an article) to bend freely and repeatably without breaking and/or to conform to the shape of the body part to which the flexible material (or article) is applied. In some embodiments, the flexible textile substrate 104 can have regions with different degrees of elasticity which can help facilitate different levels of compression on the leg of the wearer.

As shown in FIG. 1, the body 102 of the knee brace 100 can have a distal portion 106 which can surround a portion of the calf of a wearer and can be positioned below the knee K of a wearer. Additionally, the distal portion 106 can have a first end 107 and a second end 108, where the first end 107 and the second end 108 can connect or be joined at an edge to provide support or compression on the calf of the wearer by surrounding the calf of the wearer. For example, the first end 107 of the distal portion 106 and the second end 108 of the distal portion 106 can generally extend in the x-direction (relative to the illustration). The first end 107 of the distal portion 106 and the second end 108 of the distal portion 106 can be joined together with a repeatable pattern or stitching pattern. In some embodiments, the stitching pattern can be a zig-zag stitching pattern or a three needle stitching pattern, where the repeatable pattern can connect the first end 107 and second end 108 of the distal portion 106 so that the distal portion 106 can extend or horizontally wrap around the calf of a wearer.

The distal portion 106 of the body 102 can have a bottom edge 109 and a top edge 110, where the bottom edge 109 of the distal portion 106 can be designed to cover the bottom portion of a calf of the wearer and can generally extend in an x-direction around the calf. Additionally, the bottom edge 109 of the distal portion 106 also can also be at the terminal end of the body 102 of the knee brace 100. In some embodiments, the top edge 110 of the distal portion 106 can surround or encircle a bottom portion of the knee of a wearer. The top edge 110 of the distal portion 106 can follow a curvilinear path of approximately 180 degrees.

Furthermore, as shown in FIG. 1, the body 102 of the knee brace 100 can have a proximal portion 112, where the proximal portion 112 can have a top edge 113 and a bottom edge 114. The bottom edge 114 of the proximal portion 112 can surround or encircle the top portion of the knee of the wearer. The top edge 110 of the distal portion 106 and the bottom edge 114 of the proximal portion 112 can form a circle around the knee patella of the wearer, which can provide support to the knee joint and permit the knee to rotate along a sagittal plane more freely. Additionally, the top edge 113 of the proximal portion 112 can surround the thigh of the wearer. Therefore, in some embodiments, the distal portion 106 of the body 102 and the proximal portion 112 of the body 102 can encircle the entire knee of the wearer. Advantageously, this can provide the encircled portion of the knee (e.g., patella) with additional support or pressure to unload the affected areas of the knee to provide pain relief. Additionally, the proximal portion 112 can be configured to fit around various thigh sizes or thigh circumferences. For example, and without limitation, the body 102 of the knee brace 100 can be configured to fit around the thigh of a wearer, where the thigh can have a circumference of 33 cm to 75 cm, and where the circumference of the thigh can be measured approximately 15 cm above the center of the knee patella. Additionally, the knee brace 100 can be provided in a plurality of different sizes (e.g., small, medium, large, etc.) to accommodate to different leg sizes.

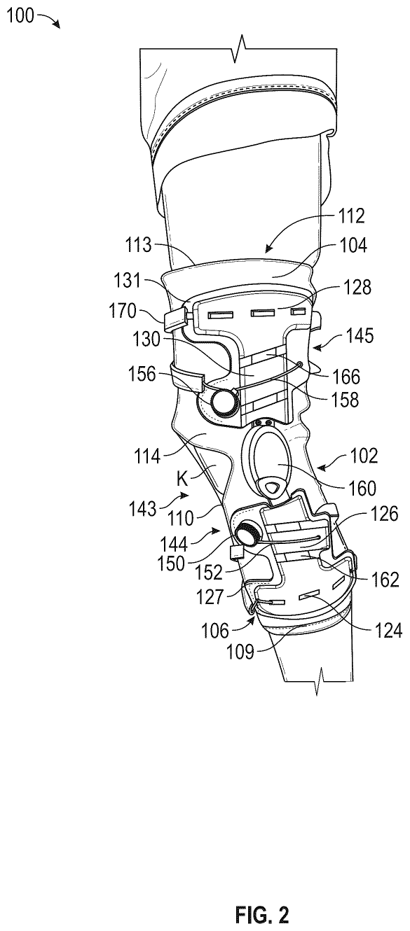

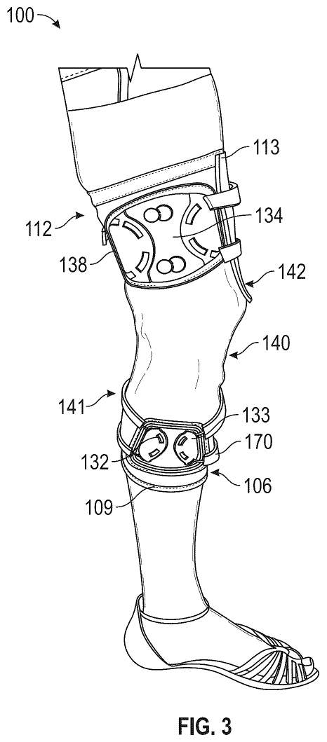

As shown in FIG. 1-4, there can be one or more supports 122 positioned on the knee brace 100. The one or more supports 122 can be positioned on the outward facing surface 116 of the knee brace 100. The one or more supports 122 can be semi-rigid (e.g., plastic, polyurethane) which can advantageously allow for the one or more supports to bend or flex to shape of the leg of the wearer. Additionally, the material of the one or more supports 122 can improve manufacturability by improving the assembly time, reliability, and/or costs of positioning the one or more supports 122 on the knee brace 100. In some embodiments, the one or more supports 122 are located on medial and lateral portions of the brace of a wearer. This can provide support to help to provide unicompartmental loading on the healthy portion of a knee instead of the affected/injured portion of a knee.

As shown in the illustrated embodiment, the one or more supports 122 can be separate, distinct (e.g., individual) supports which include a first support 124, a second support 128, a third support 132, and a fourth support 134. The first support 124, second support 128, third support 132, and fourth support 134 can all have different geometric profiles which can provide the appropriate support for the region of the body 102 the support is located. The first support 124 can be positioned laterally on the outward facing surface 116 of the distal portion 106 of the body 102. The second support 128 can be positioned laterally on the outward facing surface 116 of the proximal portion 112 of the body 102. The third support 132 can be positioned medially on the outward facing surface 116 of the distal portion 106 of the body 102. The fourth support 134 can be positioned medially on the outward facing surface 116 of the proximal portion 112 of the body 102. The one or more supports on the body 102 of the knee brace 100 can serve to strategically reinforce or support certain joint or limb structures and can facilitate or resist specific movements of the joints. The one or more supports 122 can, for example, offload certain joint structures and can be oriented to reduce loads on the medial or lateral compartments of the knee joints. In some embodiments, the one or more supports 122 can be designed in a fashion where the supports are configured to conform to the anatomical features of the wearer. For example, the first support 124 can be configured to be curved to fit on the lateral portion of the calf of the wearer. The first support 124 can be made of a semi-rigid material which can bend to the calf of the wearer to provide additional support. The second support 128 can be configured to be curved to fit on the lateral portion of the thigh of the wearer. Advantageously, the second support 128 can be made of a semi-rigid material which can bend to conform to the thigh of the wearer. The third support 132 can bend to conform to the medial portion of the calf of the wearer and the fourth support 134 can bend to conform to the medial portion of the thigh of a wearer. In some embodiments, the one or more supports can be positioned or attached to the body 102 with a repeatable pattern. Each of the one or more supports 122 can have an edge 127, 131, 133, 138, where the edge can be attached to the body 102 and/or flexible textile substrate 104 via a straight stitching pattern. Additionally, in some embodiments, the knee brace 100 can be configured such that the first support 124 and second support 128 can be positioned medially on the body 102 and the third support 132 and fourth support 134 can be positioned on the lateral portion of the body 102.

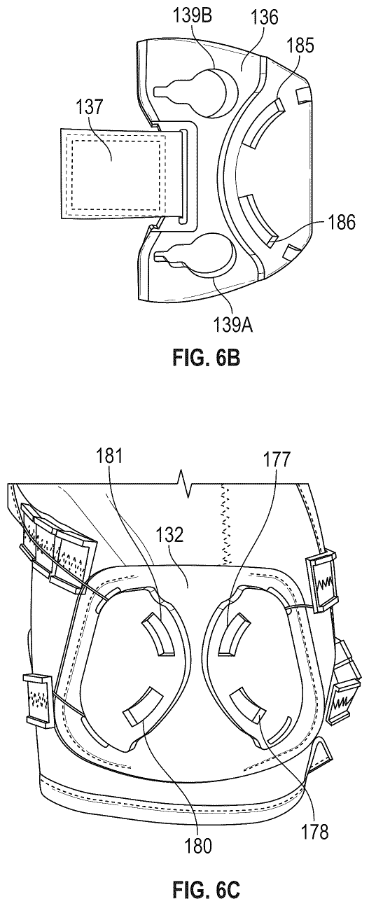

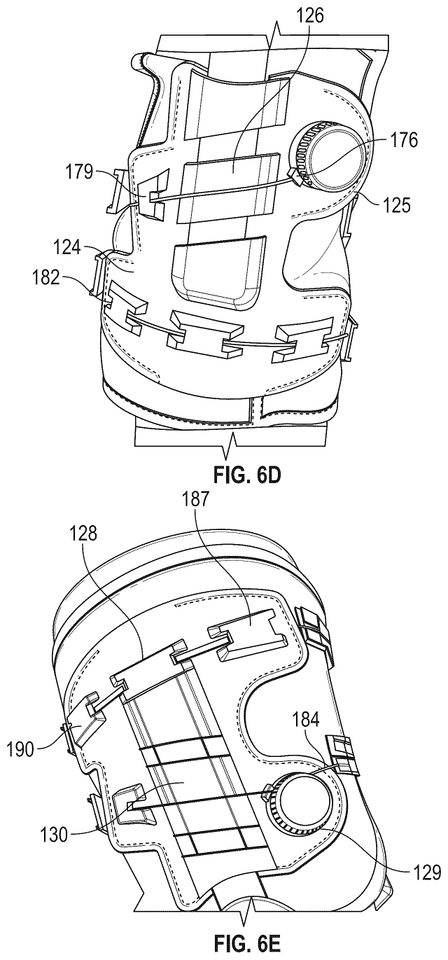

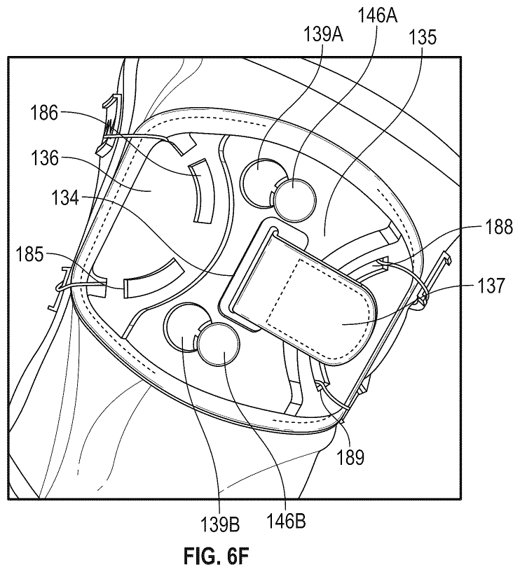

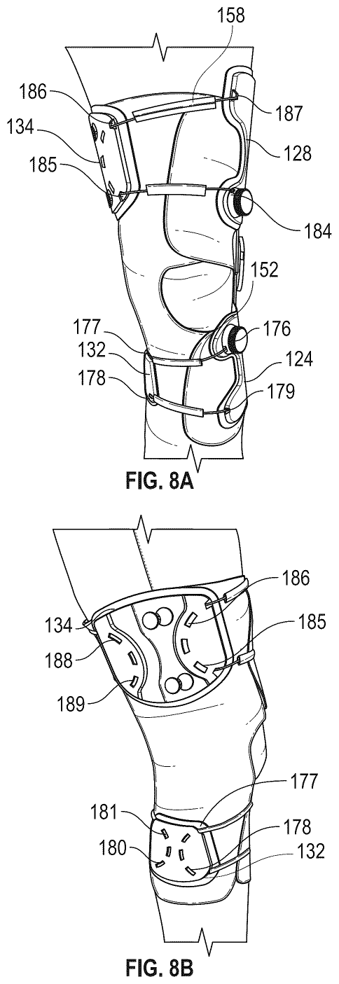

The knee brace 100 can have a first tightening mechanism 148 and a second tightening mechanism 154 (see, for example, FIG. 1). The first tightening mechanism 148 can have a first rotating dial 150 and a first cable 152. The second tightening mechanism 154 can have a second rotating dial 156 and a second cable 158. To couple the first tightening mechanism 148 to the knee brace 100, the first support 124 can include a region or opening 125 sized and configured for the first rotating dial 150 (see, for example, FIG. 6D). Alternatively, the first rotating dial 150 can be directly coupled (e.g., glued, molded) to the first support 124. Furthermore, the first tightening mechanism 148 can be coupled to the first support 124 and the third support 132, where the first cable 152 can couple or connected to the first support 124 and the third support 132 (see, for example, locations in FIGS. 8A-8C). The first tightening mechanism 148 can be configured to incrementally tension the distal portion 106 of the body 102 by increasing the tension in the first cable 152. Thus, rotating the first rotating dial 150 in a first direction can increase tension and lead to an increased compressive or biomechanical force on the distal portion 106 of the body 102 due to increased tensioning from the first support 124 and third support 132. Additionally, rotating the first rotating dial 150 in a second direction can release tension in the distal portion 106 of knee brace 100 and can release tension in the cable, which can allow the user to remove the knee brace 100 from the leg of the wearer.

The second tightening mechanism 154 can be coupled to the second support 128 and the fourth support 134, where the second support 128 and the fourth support 134 can be coupled via the second cable 158 (see, for example, locations in FIGS. 8A-8C). The second support 128 can have or include a region or opening 129 sized and configured for the second rotating dial 156 (see, for example, FIG. 6E). Alternatively, the second rotating dial 156 can be directly coupled (e.g., glued, molded) to the second support 128. The second tightening mechanism 154 can be configured to incrementally tension the proximal portion 112 of the body 102 by increasing tension in the second cable 158. In some embodiments, rotating the second rotating dial 156 in a first direction can increase the tension in the second cable 158 and increase the compressive or biomechanical forces in the proximal portion 112 of the body 102. Increasing tension in the second cable 158 can cause the second support 128 and the fourth support 134 to compress on the thigh or proximal portion of the wearer's leg. In some embodiments, rotating the second rotating dial 156 in a second direction can release tension in the proximal portion 112 of the wearer's leg by releasing tension in the second cable 158. Therefore, by rotating the second rotating dial 156 in a second direction the compressive forces exerted by the second support 128 and the fourth support 134 on the wearer's leg are reduced so that the knee brace 100 is of a desired comfort.

The knee brace 100 can have a hinge 160 located on a lateral section of the knee brace 100. The hinge 160 can, in some embodiments, be located on a lateral side of a wearer and can be configured to support movement of a knee of a wearer along a fixed path (e.g., forward/backward, sagittal plane). The hinge 160 can be coupled to the first support 124 and second support 128 via one or more assembly bars (first assembly bar 162, second assembly bar 166). In some embodiments, the hinge 160 can be coupled to a condyle pad 161, where the condyle pad 161 can be positioned between the hinge 160 and a condyle area of the knee of a wearer. In some embodiments the condyle pad 161 can be made from a compliant material which can include silicon or other similar materials (e.g., memory foam, soft foam, semi-rigid foam, polyethylene, polyurethane, etc.). Additionally, the condyle pad 161 can have different thicknesses to ensure there is no gap between the hinge 160 and the condyle of the patient. The condyle pad 161, in some embodiments, can have a thickness of between about 3 mm and 20 mm. Advantageously, the condyle pad 161 can be configured to control an amount of pressure applied by the hinge 160 to the condyle area of the knee of a wearer. The condyle pad 161 can provide a lateral load on the lateral section 143 of the knee brace 100, which can provide additional support to alleviate pain in the knee. Alternatively, in some embodiments, the hinge 160 can be located on the medial section 140 of the knee brace 100. Therefore, in some embodiments, the positioning of the knee brace 100 can be reversed with respect to the disclosure above (i.e., hinge 160 on medial section 140, first support 124 on medial section 140, second support 128 on medial section 140, third support 132 on lateral section 143, fourth support 134 on lateral section 143). Advantageously, placing the hinge 160 on the medial portion of the leg can assist a user when the need for biomechanical loading is necessary for the medial side. In these embodiments, the condyle pad 161 can provide a medial load on the medial section of the knee brace 100, which can provide additional support to alleviate pain in the knee. In some embodiments, the hinge 160 can be located on both the medial section 140 and the lateral section 143 of the knee brace 100. In these embodiments, the one or more supports 122 can be configured to couple to the hinges 160 (i.e., multiple first supports 124 and second supports 128 where the first support 124 and second supports 128 are located on the medial section 140 and lateral section 143 of the knee brace 100).

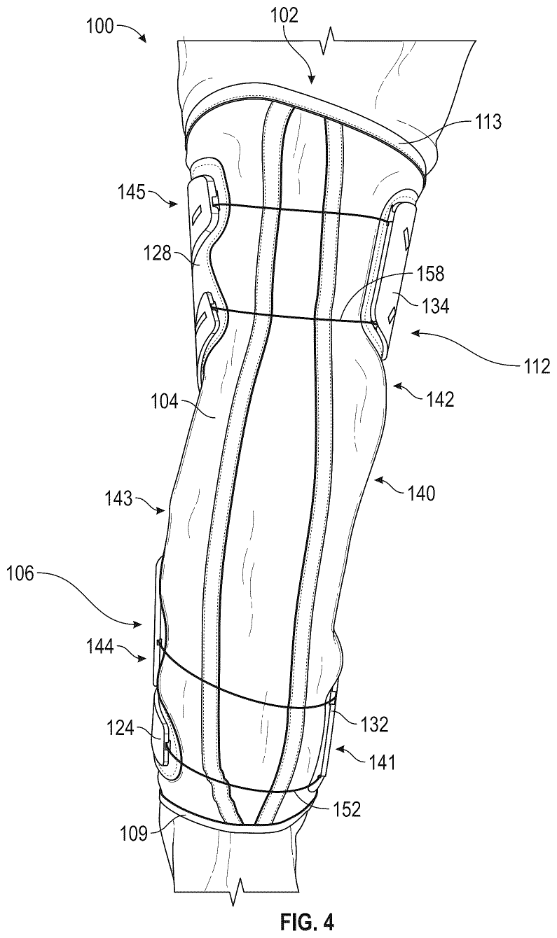

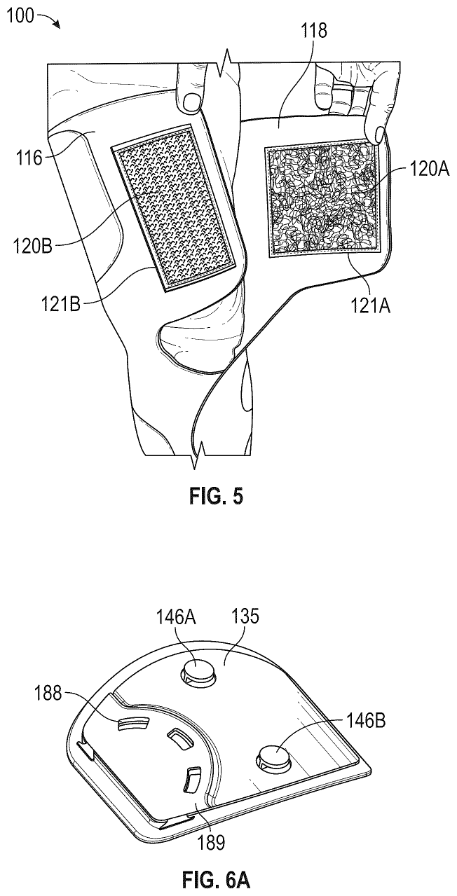

FIG. 5 shows a perspective view of the proximal portion 112 of a knee brace 100, where the knee brace 100 can have an outward facing surface 116 and an inward facing surface 118. In the embodiment illustrated in FIG. 5, the inward facing surface 118 can be configured to face in an opposite direction of the outward facing surface 116. In some embodiments disclosed herein, the body 102 can have a fastener 120A, 120B which can be configured to couple the outward facing surface 116 at the proximal portion 112 of the body 102 and the inward facing surface 118 at the proximal portion 112 of the body 102. The inward facing surface 118 can be configured to partially overlap the outward facing surface 116 and then fasten to the outward facing surface 116. The fastener 120A, 120B can be a hook and loop fastener, where the hook can be located on the outward facing surface 116 and the loop can be located on the inward facing surface 118. Alternatively, in some embodiments, the fastener can be a snap, button, clasp, etc. The fasteners 120A, 120B (e.g., hook and loop fastener) can have one or more edges 121B, 121A, which can be attached the body 102 via a repeatable pattern (i.e., zig-zag stitch, 3 needles stitching pattern).

FIGS. 6A-6F illustrate the one or more supports disposed on the body 102 of the knee brace 100. In some embodiments disclosed herein, the fourth support 134 can include a first component 135 and a second component 136, where the first component 135 can be removably coupled to the second component 136. As illustrated in FIG. 6A, the first component 135 can have one or more connectors 146A, 146B located on the surface which can be used to couple the first component 135 and the second component 136. Additionally, the first component 135 can have locations (e.g., third location 188 on fourth support 134 and fourth location 189 on fourth support 134) to guide the second cable 158 through the first component 135 (and the fourth support 134) to increase or reduce the tension in the proximal portion 112 of the knee brace 100 to support the wearer. As illustrated in FIG. 6B, the fourth support 134 can have a second component 136, which can have one or more connector openings 139A, 139B. The one or more connector openings 139A, 139B can be used to couple the first component 135 to the second component 136 via the one or more connectors 146A, 146B of the first component 135. The second component 136 can also have locations (e.g., first location 185 on the fourth support 134 and second location 186 on the fourth support 134) to guide the second cable 158 through the second component 136 (and through the fourth support 134) to increase tension in the proximal portion 112 of the knee brace 100. The second component 136 can have a pulling aid 137 to increase tension in the proximal portion 112 of the knee brace 100. The pulling aid 137 can be used to help close the fourth support 134 where, in some embodiments, the fourth support 134 includes the first component 135 and the second component 136. As illustrated in FIG. 6C, the third support 132 can have one or more locations (e.g., first location 177, second location 178, third location 180, fourth location 181) which can guide the first cable 152 though the third support 132 to increase or reduce tension in the distal portion 106 of the knee brace 100. As illustrated in FIG. 6D, the first support 124 can have one or more locations (e.g., first location 176, second location 179, third location 182) which can guide the first cable 152 through the first support 124. In some embodiments, the first support 124 can have an opening or first slot which can support the hinge 160 (e.g., hinge assembly). As illustrated in FIG. 6E, the second support 128 can also have one or more locations (e.g., first location 184, second location 187, third location 190) which can guide the second cable 158 though the second support 128, which increase or reduce tension in the proximal region of the wearer. The second support 128 can have an opening or a second slot which can support the hinge 160. FIG. 6E is another embodiment of the fourth support 134, wherein the first component 135 and second component 136 are combined. By using the pulling aid 137 the wearer can increase tension in the proximal portion 112 of the knee brace 100. The pulling aid 137 can be used to help close the fourth support 134. In some embodiments, the first component 135 and the second component 136 can create a closure system, where the closure system allows the user to tighten or loosen the brace to the particular needs of the wearer (e.g., pain relief, removing the brace, wearing the brace, preventing the brace from slipping). In some embodiments the closure system can assist in unicompartmental loading of the knee.

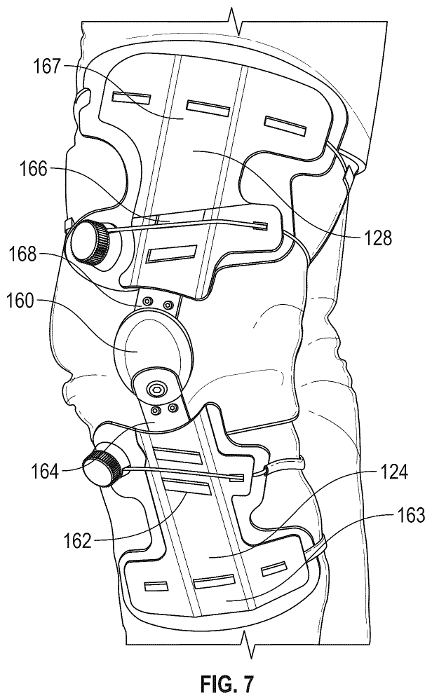

FIG. 7 illustrates a lateral portion of the knee brace 100, further illustrating the hinge. For example, the hinge 160 can be located on a lateral section 143 of the knee brace 100 and can be configured to support movement of the knee of the wearer. The hinge 160 can have a first assembly bar 162 and a second assembly bar 166. The first assembly bar 162 can have a distal end 163 and a proximal end 164 which can be coupled to the knee brace 100 (e.g., screw, bolt, glue, stitched, etc.). Additionally, the second assembly bar 166 can have a distal end 167 and a proximal end 168 which can be configured to couple to the knee brace 100. In some embodiments, a proximal end 164 of the first assembly bar 162 and a proximal end 168 of the second assembly bar 166 can be coupled to the hinge 160. Furthermore, in some embodiments, the distal end 163 of the first assembly bar 162 can slide into a slot 126 of the first support 124 and a distal end 167 of the second assembly bar 166 can slide into a slot 130 of the second support 128. By coupling to the first support 124 and second support 128, the hinge 160 can allow the knee of the user to move along the sagittal plane. Furthermore, the hinge 160 can absorb lateral biomechanical forces to help unload affected portions of the knee to alleviate pain. In some embodiments, the hinge 160 can help unload portions of the knee by distributing load to the first support 124 and/or second support 128 to further distribute load along the leg of the wearer.

FIGS. 8A-8C illustrate the path of the first cable 152 and second cable 158, where the first cable 152 can connect the first support 124 and third support 132 to provide tension in a distal region of the wearer, and the second cable 158 can connect the second support 128 and the fourth support 134 to provide tension in a proximal region of the knee brace 100. The path of the first cable 152 can include the first cable 152 starting at a first location 176 on the first support 124 and traveling to a first location 177 on the third support 132, then to a second location 178 on the third support 132, then to a second location 179 on the first support 124, then to a third location 180 on the third support 132, then to a fourth location 181 on the third support 132, then to a third location 182 on the first support 124 before returning to the first location 176 on the first support 124. Advantageously, the first cable 152 does not intersect, bisect, or cross over any portion of itself (e.g., first cable 152). The first cable 152 goes through the specified locations (e.g., channels) to follow a particular path to provide compression to the distal portion 106 of the knee brace 100.

Additionally, the path of the second cable 158 can start at a first location 184 on the second support 128 and travel to a first location 185 on the fourth support 134, then to a second location 186 on the fourth support 134, then to a second location 187 of the second support 128, then to a third location 188 on the fourth support 134, then to a fourth location 189 on the fourth support 134, then to a third location 190 on the second support 128, before returning to the first location 184 on the second support 128. Advantageously, the second cable 158 does not intersect, bisect, or cross over any portion of itself (e.g., second cable 158). The second cable 158 goes through the specified locations (e.g., channels) to follow a particular path to provide compression to the proximal portion 112 of the knee brace 100. In some embodiments, the length of the first cable 152 can between 70 cm and 120 cm based on the size or requirements of the wearer and the length of second cable 158 can be between 80 cm and 210 cm.

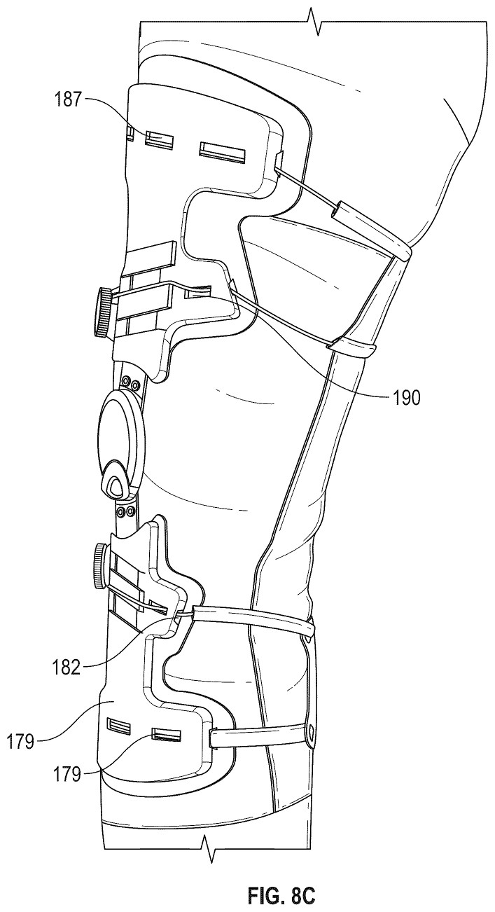

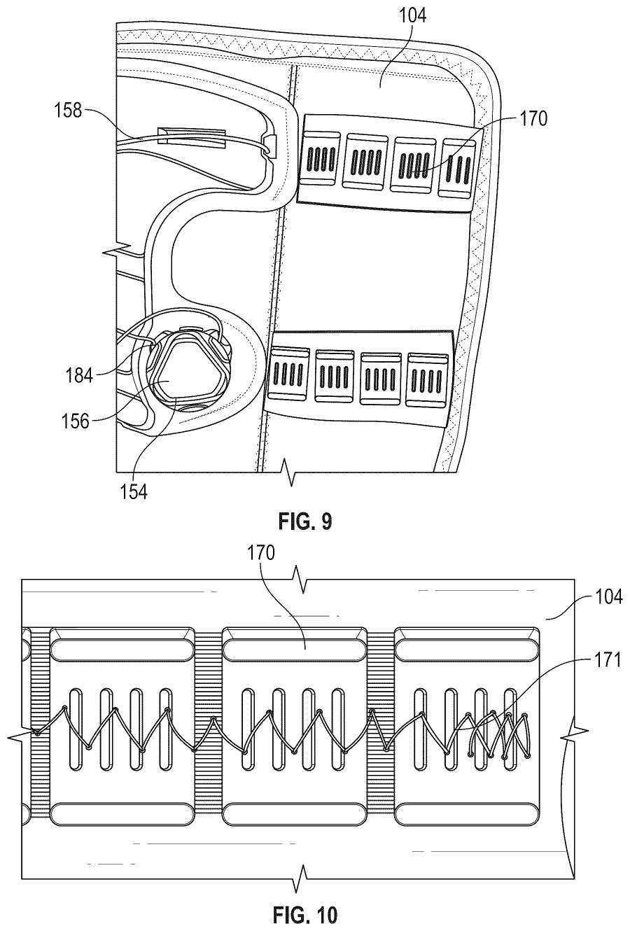

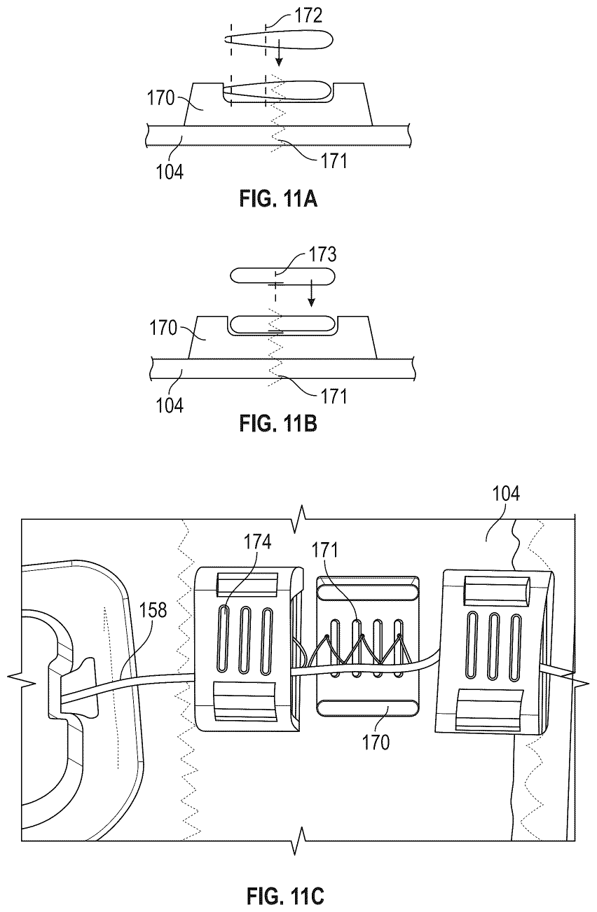

FIGS. 9 and 10 illustrate that the first tightening mechanism 148 and/or the second tightening mechanism 154 can include one or more cable guides 170. As shown in FIG. 11A, the one or more cable guides 170 can include one or more sided closures 172 stitched into the one or more cable guides 170 to retain the first cable 152 or second cable 158. As shown in FIG. 11B, the one or more cable guides 170 can include one or more hidden closures 173 which can be stitched 171 into the one or more cable guides 170 to retain the first cable 152 or second cable 158. Additionally, as shown in FIG. 11C, the one or more cable guides 170 can include one or more clamps 174 where the one or more clamps can be removably mounted to the one or more cable guides to retain the second cable 158. In some embodiments, the one or more cable guides can be fastened to the body 102 with a zig-zag sewing pattern.

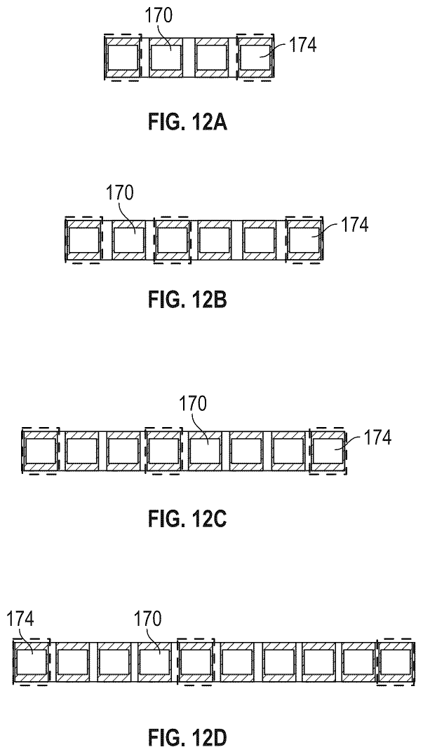

FIGS. 12A-12D illustrate embodiments of the knee brace 100 where the clamps 174 on the one or more cable guides 170 can be located at various positions. For example, and without limitation, as shown in FIG. 12A, if there are less than or equal to four cable guides 170, the one or more clamps 174 (e.g., plastic clamps) are located at the extremities of the series of cable guides 170. As shown in FIG. 12B if there are more than four, but less than or equal to six cable guides 170, then there are clamps 174 at the extremities of the series of cable guides 170 and there is a clamp 174 located at every third increment of cable guides 170. For example, and without limitation, as shown in FIG. 12C, if there are more than six but less than or equal to eight cable guides 170, then there can be clamps 174 located at the extremities of the series of cable guides 170 and clamps 174 at every fourth increment of the series of cable guides 170. For example, and without limitation, as shown in FIG. 12D, if there are more than eight clamps 174, but less than or equal to ten clamps 174, there can be clamps 174 located at the extremities of the one or more cable guides 170 and there can be clamps at every fifth increment of the series of cable guides 170.

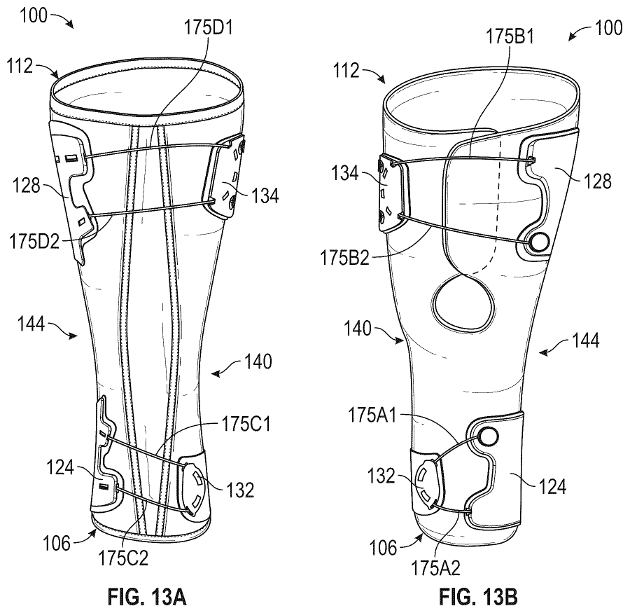

FIGS. 13A and 13B illustrate a front view and a rear view of the knee brace 100, where the one or more cable guides 170 can be (although not shown) positioned on the distal portion 106 and the proximal portion 112 of the knee brace 100. The one or more cable guides 170 can be positioned in one or more areas. The one or more areas can include an upper first position 175A1, a lower first position 175A2, an upper second position 175B1, a lower second position 175B2, an upper third position 175C1, a lower third position 175C2, an upper fourth position 175D1, and a lower fourth position 175D1. In some embodiments, the upper and lower first 175A1, 175A2 positions can cover the first cable 152 which can be coupled to the first support 124 and the third support 132. Additionally, the upper and lower second positions 175A1, 175A2 can cover the second cable 158 which can be coupled to the second support 128 and the fourth support 134. In some embodiments, the upper and lower third positions 175C1, 175C2 can cover the first cable 152 which can be coupled to the first support 124 and third support 132. The upper and lower fourth positions 175D1, 175D2 can cover the second cable 158 which can be coupled to the second support 128 and the fourth support 134.

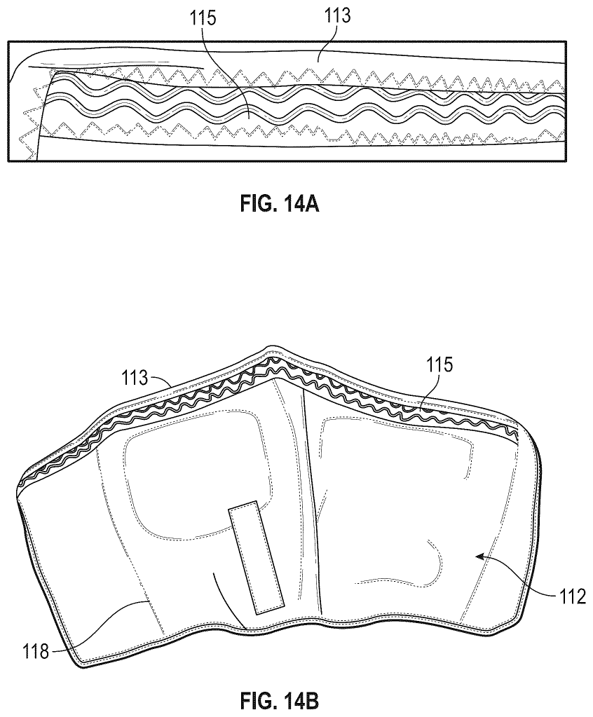

FIGS. 14A and 14B illustrate the body 102 of the knee brace 100, which can have an anti-slip portion 115 is located on the inward facing surface 118 of the proximal portion 112. The anti-slip portion 115 can be located at the top edge of the proximal portion 112 of the body 102. The anti-slip portion 115 can allow the proximal portion 112 of the knee brace 100 to remain on an upper portion of the leg of the wearer. During use a wearer may perspire or the clothing layered between the knee brace 100 and the wearer may become slippery, which can cause the brace to move out of position. Therefore, by having the anti-slip portion 115, the knee brace 100 can remain in position to approximately provide biomechanical loading to the wearer's knee.

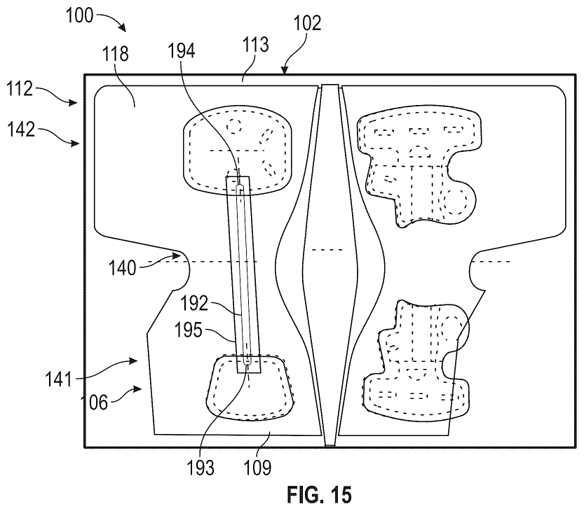

FIG. 15 illustrates an inward facing surface 118 of the body 102, where the inward facing surface 118 can have a lateral flexible stay 192 positioned on a medial portion of the body 102. The lateral flexible stay 192 can provide additional support to the medial side of the knee of a wearer. Therefore, the affected knee can have pressure applied to a medial portion of the knee to alleviate the pain affected in the unicompartmentally affected region of the knee. In some embodiments, the lateral flexible stay 192 can have a first end 193 and a second end 194. The first end of the lateral flexible stay 192 can extend to a position connectable to the third support 132. Additionally, the second end 194 of the lateral flexible stay 192 can extend to a position connectable to the fourth support 134. To hold the lateral flexible stay 192 in position, the body 102 can have a cover 195 positioned on the inward facing surface 118 of the body 102. By placing the lateral flexible stay 192 within the cover 195, the lateral flexible stay 192 can be coupled to the body 102. Additionally, the cover 195 can extend from the third support 132 to the fourth support 134. In some embodiments disclosed herein, the lateral flexible stay 192 is made of flexible steel. Advantageously, the lateral flexible stay 192 can have a modulus of elasticity which permits the lateral flexible stay 192 to bend when the knee applies a medial load to the lateral flexible stay 192, but also provides a sufficient counterforce to the medial region of the knee to provide adequate support. Depending on the size of the wearer, the lateral flexible stay 192 can be about 220 mm. However, in some embodiments, the lateral flexible stay 192 can be between 150 mm and 300 mm.

In some embodiments, the inward facing surface 118 can have a lateral flexible stay 192 positioned on a lateral portion of the body 102. The lateral flexible stay 192 can provide additional support to the lateral side of the knee of a wearer. Advantageously, the lateral flexible stay 192 is positioned on the affected side of the knee of the wearer (e.g., medial side and/or lateral side). Therefore, the affected knee can have pressure applied to a lateral portion of the knee to alleviate the pain in the unicompartmentally affected region of the knee (for example, a single compartment affected region of the knee).

In some embodiments, a method for providing stability to a knee joint is disclosed. The method can include using embodiments of the knee brace 100 illustrated in FIGS. 1-15. To use the knee brace 100 a user will first, position the distal portion 106 of the flexible textile substrate 104 around the calf of the wearer but keep the distal portion 106 below the knee of the wearer. Furthermore, utilizing the knee brace 100 will include positioning the proximal portion 112 of the flexible textile substrate 104 around the thigh of the wearer and keeping the proximal portion 112 above the knee of the wearer. Next the user will need to position the proximal portion 112 of the flexible textile substrate 104 and the distal portion 106 of the flexible textile substrate 104 around the knee of the wearer, ensuring that the distal portion 106 and proximal portion 112 are encircling a patellar region of the wearer's knee.

The brace can be positioned, such that the first support 124 and the second support 128 are located a lateral side of the wearer and the third support 132 and the fourth support 134 are located a medial side of the wearer. However, in some alternative embodiments, the first support 124 and second support 128 may be located on the medial section 140 of the knee brace 100 and the third support 132 and fourth support 134 may be located on the lateral side 143 of the knee brace 100. To ensure the knee brace 100 is secure at the distal portion 106, the user will tighten the first tightening mechanism 148, which will incrementally tension the distal portion 106 of the flexible textile substrate 104. Tightening the first tightening mechanism 148 includes rotating the first rotating dial 150 in a first direction to increase tension in the first cable 152. To ensure the knee brace 100 is secure at the proximal portion 112, the user can tighten the second tightening mechanism 154, which will incrementally tension the proximal portion 112 of the flexible textile substrate 104. Tightening the second tightening mechanism 154 includes rotating the second rotating dial 156 in a first direction to increase tension in the second cable 158 which can provide support to the knee area of a wearer. Furthermore, loosening the first tightening mechanism 148 to release tension in the distal portion 106 can include releasing tension in the first cable 152 by rotating the first rotating dial 150 in a second direction or releasing the first rotating dial 150. Loosening the second tightening mechanism 154 includes releasing tension in the second rotating dial 156 by rotating the second rotating dial 156 in a second direction or by releasing the second rotating dial 156. By loosening both the first tightening mechanism 148 and the second tightening mechanism 154 the user can remove the knee brace 100 or release tension in the brace. In some embodiments, the hinge 160 is positioned on a lateral side of the wearer and the hinge 160 assembly can be configured to allow the knee to flex and extend on a sagittal plane. However, in alternative embodiments, the hinge 160 is positioned on a medial side of the wearer or the medial side of the knee.

To stabilize the knee joint, tightening the first tightening mechanism 148 incrementally can tension the distal portion 106 of the body 102. Additionally, tightening the first cable 152 of the first tightening mechanism 148, which can extend from a first location 176 on the first support 124 to a first location 177 on the third support 132, to a second location 178 on the third support 132 to a second location 179 on the first support 124, to a third location 180 on the third support 132 to a fourth location 181 on the third support 132, to a third location of the first support 124 to the first location at the first support 124 can increase the tension on a distal portion 106 of the knee brace 100. Furthermore, tightening the second tightening mechanism 154 can incrementally tensions the proximal portion 112. Tightening the second cable 158 which can extend from a first location on the second support 128 to a first location on the fourth support 134, to a second location on the fourth support 134 to a second location of the second support 128, to a third location on the fourth support 134 to a fourth location on the fourth support 134, to a third location on the second support 128 to the first location on the second support 128 can increase tension on a proximal portion 112 of the knee brace 100.

### ADDITIONAL EMBODIMENTS

In embodiments of the present disclosure, devices and methods for relieving pressure on a portion of a knee may be in accordance with any of the following clauses:

Clause 1. A knee brace, comprising: a body comprising a flexible textile substrate, the body comprising a distal portion configured to surround a portion of a calf of a wearer and extend below a knee of the wearer; a proximal portion configured to surround a portion of a thigh of the wearer and extend above the knee of the wearer; an outward facing surface of the proximal portion and the distal portion; an inward facing surface of the proximal portion and the distal portion configured to face an opposite direction of the outward facing surface; and a fastener configured to couple the outward facing surface of the proximal portion and the inward facing surface of the proximal portion; one or more supports, the one or more supports comprising: a first support positioned laterally on the outward facing surface of the distal portion of the body; a second support positioned laterally on the outward facing surface of the proximal portion of the body; a third support positioned medially on the outward facing surface of the distal portion of the body; and a fourth support positioned medially on the outward facing surface of the proximal portion of the body; a first tightening mechanism comprising a first rotating dial and a first cable, the first cable coupled to the first support and the third support; a second tightening mechanism comprising a second rotating dial and a second cable, the second cable coupled to the second support and the fourth support; and a hinge positioned laterally on the knee brace and coupled to the first support and the second support; wherein the distal portion of the body and the proximal portion of the body are configured to encircle the knee of the wearer; wherein the first tightening mechanism is configured to incrementally tension the distal portion by rotating the first rotating dial to increase the tension in the first cable and is configured to release tension in the distal portion by releasing the tension in the first cable; wherein the second tightening mechanism is configured to incrementally tension the proximal portion by rotating the second rotating dial to increase the tension in the second cable and is configured to release tension in the proximal portion by releasing the tension in the second cable.

Clause 2. The knee brace of clause 1, wherein the distal portion comprises a first end and a second end, the first end and the second end are coupled together with a repeatable pattern.

Clause 3. The knee brace of clause 2, wherein the repeatable pattern is a zig-zag sewing pattern.

Clause 4. The knee brace of any of clauses 1-3, wherein the one or more supports are positioned or attached to the body with a repeatable pattern.

Clause 5. The knee brace of clause 4, wherein the repeatable pattern is a zig-zag sewing pattern.

Clause 6. The knee brace of any of clauses 1-5, wherein the one or more supports are semi-rigid.

Clause 7. The knee brace of any of clauses 1-6, wherein the first support comprises an opening sized and configured for the first rotating dial.

Clause 8. The knee brace of any of clauses 1-7, wherein the second support comprises an opening sized and configured for the second rotating dial.

Clause 9. The knee brace of any of clauses 1-8, wherein the fourth support comprises a first component and a second component, wherein the first component is removably coupled to the second component.

Clause 10. The knee brace of clause 9, wherein the second component comprises a pulling aid, wherein the pulling aid is coupled to the second component.

Clause 11. The knee brace of clause 10, wherein the pulling aid provides increased tension to the proximal portion.

Clause 12. The knee brace of any of clauses 1-11, wherein the flexible textile substrate is made of a soft material.

Clause 13. The knee brace of any of clauses 1-12, wherein the flexible textile substrate is made of an elastomeric material.

Clause 14. The knee brace of any of clauses 1-13, wherein the flexible textile substrate is most elastic when tension is applied in a lateral direction.

Clause 15. The knee brace of any of clauses 1-14, wherein the first rotating dial is positioned on the first support.

Clause 16. The knee brace of any of clauses 1-15, wherein the second rotating dial is positioned on the second support.

Clause 17. The knee brace of any of clauses 1-16, wherein the first tightening mechanism comprises one or more cable guides.

Clause 18. The knee brace of clause 17, wherein the one or more cable guides comprises one or more sided closures stitched into the one or more cable guides to retain the first cable.

Clause 19. The knee brace of clause 17, wherein the one or more cable guides comprises one or more hidden closures stitched into the one or more cable guides to retain the first cable.

Clause 20. The knee brace of clause 17, wherein the one or more cable guides comprises one or more clamps, the one or more clamps are removably mounted onto the one or more cable guides to retain the first cable.

Clause 21. The knee brace of any of clauses 17-20, wherein the one or more cable guides are fastened to the body.

Clause 22. The knee brace of any of clauses 1-21, wherein the first cable extends from a first location on the first support to a first location on the third support, to a second location on the third support to a second location on the first support, to a third location on the third support to a fourth location on the third support, to a third location of the first support to the first location at the first support.

Clause 23. The knee brace of any of clauses 1-22, wherein the first cable between 80 cm and 110 cm.

Clause 24. The knee brace of any of clauses 1-23, wherein the second cable extends from a first location on the second support to a first location on the fourth support, to a second location on the fourth support to a second location of the second support, to a third location on the fourth support to a fourth location on the fourth support, to a third location on the second support to the first location on the second support.

Clause 25. The knee brace of any of clauses 1-24, wherein the second cable is between 90 cm and 190 cm.

Clause 26. The knee brace of any of clauses 1-25, wherein the second tightening mechanism comprises one or more cable guides.

Clause 27. The knee brace of clause 26, wherein the one or more cable guides comprises one or more sided closures stitched into the one or more cable guides to retain the second cable.

Clause 28. The knee brace of clause 26, wherein the one or more cable guides comprises one or more hidden closures stitched into the one or more cable guides to retain the second cable.

Clause 29. The knee brace of clause 26, wherein the one or more cable guides comprises one or more clamps, the one or more clamps are removably mounted onto the one or more cable guides to retain the second cable.

Clause 30. The knee brace of clause 26, wherein the one or more cable guides are fastened to the body, the one or more cable guides are fastened to the body with a zig-zag sewing pattern.

Clause 31. The knee brace of any of clauses 1-30, wherein the hinge is located on a lateral side of the knee of the wearer and configured to support movement of the knee of the wearer.

Clause 32. The knee brace of any of clauses 1-32, wherein the hinge comprises a first assembly bar and a second assembly bar, the first assembly bar having a distal end and a proximal end, the second assembly bar having a distal end and a proximal end, wherein the proximal end of the first assembly bar and the proximal end of the second assembly bar are coupled to the hinge.

Clause 33. The knee brace of clause 32, wherein the distal end of the first assembly bar extends into a portion of the first support and the distal end of the second assembly bar extends into a portion of the second support.

Clause 34. The knee brace of any of clauses 32-33, wherein the first assembly bar engages with a first slot located on the first support, wherein the second assembly bar engages with a second slot located on the second support.

Clause 35. The knee brace of any of clauses 1-34, wherein the one or more supports are coupled to the body with a repeatable pattern.

Clause 36. The knee brace of clause 35, wherein the repeatable pattern is a zig-zag sewing pattern.

Clause 37. The knee brace of any of clauses 1-36, wherein the fastener is a hook and loop fastener.

Clause 38. The knee brace of clause 37, wherein the fastener is connected to the body with a repeatable pattern, the repeatable pattern is a zig-zag sewing pattern.

Clause 39. The knee brace of any of clauses 1-38, further comprising an anti-slip portion, the anti-slip portion is located on the inward facing surface of the proximal portion.

Clause 40. The knee brace of any of clauses 1-39, further comprising a condyle pad, wherein the condyle pad is located between the hinge and a condyle area of the knee of a wearer.

Clause 41. The knee brace of clause 40, wherein the condyle pad is a compliant material.

Clause 42. The knee brace of any of clauses 40-41, wherein the condyle pad is a thickness of between about 5 mm and about 18 mm.

Clause 43. The knee brace of any of clauses 40-42, wherein the condyle pad is configured to control an amount of pressure applied by the hinge to the condyle area of the knee of a wearer.

Clause 44. The knee brace of any of clauses 1-44, further comprising a lateral flexible stay, wherein the lateral flexible stay is positioned on a medial portion of the body.

Clause 45. The knee brace of clause 44, wherein the lateral flexible stay comprises a first end and a second end, the first end extends to a position connectable to the third support, the second end extends to a position connectable to the fourth support.

Clause 46. The knee brace of any of clauses 44-45, wherein the body comprises a cover, the cover is positioned on the inward facing surface of the body.

Clause 47. The knee brace of clause 46, wherein the lateral flexible stay is coupled to the cover.

Clause 48. The knee brace of clause 47, wherein the cover extends from the third support to the fourth support.

Clause 49. The knee brace of any of clauses 44-48, wherein the lateral flexible stay is made of flexible steel.

Clause 50. The knee brace of any of clauses 44-49, wherein the lateral flexible stay is between approximately 200 mm and approximately 240 mm.

Clause 51. The knee brace of any of clauses 1-50, further comprising a lateral flexible stay, wherein the lateral flexible stay is positioned on a lateral portion of the body.

Clause 52. A knee brace comprising: a flexible textile substrate, the flexible textile substrate comprising: a distal portion configured to surround a portion of a calf of a wearer and extend below a knee of the wearer; a proximal portion configured to surround a portion of a thigh of the wearer and extend above the knee of the wearer; one or more supports, the one or more supports comprising: a first support positioned laterally on the distal portion of the flexible textile substrate; a second support positioned laterally on the proximal portion of the flexible textile substrate; a third support positioned medially on the distal portion of the flexible textile substrate; and a fourth support positioned medially on the proximal portion of the flexible textile substrate; and a hinge assembly positioned laterally on the knee brace and coupled to the first support and the second support, the hinge assembly comprising: a body; a first assembly bar having a distal end and a proximal end, wherein the proximal end is coupled to the body; and a second assembly bar having a distal end and a proximal end, wherein the proximal end is coupled to the body; wherein the fourth support comprises a first component and a second component, first and second component are removably coupled to each other.

Clause 53. The knee brace of clause 52, further comprising one or more tightening mechanisms.

Clause 54. The knee brace of clause 53, wherein the one or more tightening mechanisms is a first tightening mechanism and a second tightening mechanism, the first tightening mechanism comprises a first rotating dial and a first cable, the first cable is coupled to the first support and the third support, the second tightening mechanism comprises a second rotating dial and a second cable, the second cable is coupled to the second support and the fourth support.

Clause 55. A method for providing stability to a knee joint, comprising: providing the knee brace in clause 54; positioning the distal portion of the flexible textile substrate around the calf of the wearer and below the knee of the wearer; positioning the proximal portion of the flexible textile substrate around the thigh of the wearer and above the knee of the wearer; positioning the proximal portion of the flexible textile substrate and the distal portion of the flexible textile substrate around the knee of the wearer; positioning the first support and the second support on a lateral side of the wearer; positioning the third support and the fourth support on a medial side of the wearer; tightening the first tightening mechanism to incrementally tension the distal portion of the flexible textile substrate by rotating the first rotating dial to increase tension in the first cable; tightening the second tightening mechanism to incrementally tension the proximal portion by rotating the second rotating dial to increase tension in the second cable; loosening the first tightening mechanism to release tension in the distal portion by releasing tension in the first cable; and loosening the second tightening mechanism to release tension in the proximal portion by releasing tension in the second cable.

Clause 56. The method of clause 55, wherein the hinge assembly is positioned on a lateral side of the wearer.

Clause 57. The method of any of clauses 55-56, wherein the hinge assembly allows the knee to flex and extend on a sagittal plane.

Clause 58. The method of any of clauses 55-57, wherein tightening the first tightening mechanism incrementally tensions the distal portion when the first cable extends from a first location on the first support to a first location on the third support, to a second location on the third support to a second location on the first support, to a third location on the third support to a fourth location on the third support, to a third location of the first support to the first location at the first support.

Clause 59. The method of any of clauses 55-58, wherein tightening the second tightening mechanism incrementally tensions the proximal portion when the second cable extends from a first location on the second support to a first location on the fourth support, to a second location on the fourth support to a second location of the second support, to a third location on the fourth support to a fourth location on the fourth support, to a third location on the second support to the first location on the second support.

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the invention should not be taken to imply that the terminology is being re-defined herein to be restricted to including any specific characteristics of the features or aspects of the technology with which that terminology is associated.

It will be appreciated by those skilled in the art that various modifications and changes may be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the figures may be combined, interchanged or excluded from other embodiments.

As is also stated above, it should be noted that the use of particular terminology when describing certain features or aspects should not be taken to imply that the terminology is being re-defined herein to be restricted to including any specific characteristics of the features or aspects of the technology with which that terminology is associated. Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Headings are included herein for reference and to aid in locating various sections. These headings are not intended to limit the scope of the concepts described with respect thereto. Such concepts may have applicability throughout the entire specification.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The phrase “based on” does not mean “based only on,” unless expressly specified otherwise. In other words, the phrase “based on” describes both “based only on” and “based at least on.” Unless otherwise explicitly stated, articles such as “a” or “an” should generally be interpreted to include one or more described items. Accordingly, phrases such as “a device configured to” are intended to include one or more recited devices.

It will be appreciated by those skilled in the art that various modifications and changes may be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the figures may be combined, interchanged or excluded from other embodiments.

The above description also discloses methods and materials of the present application. The knee brace described herein may be susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims. Applicant reserves the right to submit claims directed to combinations and sub-combinations of the disclosed inventions that are believed to be novel and non-obvious. Inventions embodied in other combinations and sub-combinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

What is claimed is:

1. A knee brace, comprising:
a body comprising a flexible textile substrate, the body comprising
a distal portion configured to surround a portion of a calf of a wearer and extend below a knee of the wearer;
a proximal portion configured to surround a portion of a thigh of the wearer and extend above the knee of the wearer;
an outward facing surface of the proximal portion and the distal portion;
an inward facing surface of the proximal portion and the distal portion configured to face an opposite direction of the outward facing surface; and
a fastener configured to couple the outward facing surface of the proximal portion and the inward facing surface of the proximal portion;
one or more supports, the one or more supports comprising:
a first support positioned laterally on the outward facing surface of the distal portion of the body;
a second support positioned laterally on the outward facing surface of the proximal portion of the body;
a third support positioned medially on the outward facing surface of the distal portion of the body; and
a fourth support positioned medially on the outward facing surface of the proximal portion of the body;
a first tightening mechanism comprising a first rotating dial and a first cable, the first cable coupled to the first support and the third support;
a second tightening mechanism comprising a second rotating dial and a second cable, the second cable coupled to the second support and the fourth support; and
a hinge positioned laterally on the knee brace and coupled to the first support and the second support;
wherein the distal portion of the body and the proximal portion of the body are configured to encircle the knee of the wearer;
wherein the first tightening mechanism is configured to incrementally tension the distal portion by rotating the first rotating dial to increase the tension in the first cable and is configured to release tension in the distal portion by releasing the tension in the first cable;
wherein the second tightening mechanism is configured to incrementally tension the proximal portion by rotating the second rotating dial to increase the tension in the second cable and is configured to release tension in the proximal portion by releasing the tension in the second cable.

2. The knee brace of claim 1, wherein the one or more supports are semi-rigid.

3. The knee brace of claim 1, wherein the first support comprises an opening sized and configured for the first rotating dial, wherein the second support comprises an opening sized and configured for the second rotating dial, wherein the first rotating dial is positioned within the opening of the first support and the second rotating dial is positioned within the opening of the second support.

4. The knee brace of claim 1, wherein the fourth support comprises a first component and a second component, wherein the first component is removably coupled to the second component.

5. The knee brace of claim 4, wherein the second component comprises a pulling aid, wherein the pulling aid is coupled to the second component, wherein the pulling aid provides increased tension to the proximal portion.

6. The knee brace of claim 1, wherein the flexible textile substrate is made of an elastomeric or soft material, the flexible textile substrate is most elastic when tension is applied in a lateral direction.

7. The knee brace of claim 1, wherein the first tightening mechanism comprises one or more cable guides, the one or more cable guides comprise one or more sided closures stitched into the one or more cable guides to retain the first cable.

8. The knee brace of claim 1, wherein the first tightening mechanism comprises one or more cable guides, the one or more cable guides comprises one or more clamps, the one or more clamps are removably mounted onto the one or more cable guides to retain the first cable.

9. The knee brace of claim 1, wherein the first cable extends from a first location on the first support to a first location on the third support, to a second location on the third support to a second location on the first support, to a third location on the third support to a fourth location on the third support, to a third location of the first support to the first location at the first support.

10. The knee brace of claim 1, wherein the second cable extends from a first location on the second support to a first location on the fourth support, to a second location on the fourth support to a second location of the second support, to a third location on the fourth support to a fourth location on the fourth support, to a third location on the second support to the first location on the second support.

11. The knee brace of claim 1, wherein the hinge is located on a lateral side of the knee of the wearer and configured to support movement of the knee of the wearer, wherein the hinge allows the knee to flex and extend on a sagittal plane.

12. The knee brace of claim 1, wherein the hinge comprises a first assembly bar and a second assembly bar, the first assembly bar having a distal end and a proximal end, the second assembly bar having a distal end and a proximal end, wherein the proximal end of the first assembly bar and the proximal end of the second assembly bar are coupled to the hinge, wherein the distal end of the first assembly bar extends into a portion of the first support and the distal end of the second assembly bar extends into a portion of the second support.

13. The knee brace of claim 1, further comprising an anti-slip portion, the anti-slip portion is located on the inward facing surface of the proximal portion.

14. The knee brace of claim 1, further comprising a condyle pad, wherein the condyle pad is located between the hinge and a condyle area of the knee of a wearer, wherein the condyle pad is compliant and configured to control an amount of pressure applied by the hinge to the condyle area of the knee of a wearer.

15. The knee brace of claim 1, further comprising a lateral flexible stay, wherein the lateral flexible stay is positioned on a medial portion of the body, the lateral flexible stay comprises a first end and a second end, the first end extends to a position connectable to the third support, the second end extends to a position connectable to the fourth support.

16. The knee brace of claim 15, wherein the lateral flexible stay is made of flexible steel.

17. A knee brace comprising:
a flexible textile substrate, the flexible textile substrate comprising:
a distal portion configured to surround a portion of a calf of a wearer and extend below a knee of the wearer;
a proximal portion configured to surround a portion of a thigh of the wearer and extend above the knee of the wearer;
one or more supports, the one or more supports comprising:
a first support positioned laterally on the distal portion of the flexible textile substrate;
a second support positioned laterally on the proximal portion of the flexible textile substrate;
a third support positioned medially on the distal portion of the flexible textile substrate; and
a fourth support positioned medially on the proximal portion of the flexible textile substrate; and
a hinge assembly positioned laterally on the knee brace and coupled to the first support and the second support, the hinge assembly comprising:
a body;
a first assembly bar having a distal end and a proximal end, wherein the proximal end is coupled to the body; and
a second assembly bar having a distal end and a proximal end, wherein the proximal end is coupled to the body;
wherein the fourth support comprises a first component and a second component, first and second component are removably coupled to each other.

18. The knee brace of claim 17, further comprising one or more tightening mechanisms.

19. The knee brace of claim 18, wherein the one or more tightening mechanisms is a first tightening mechanism and a second tightening mechanism, the first tightening mechanism comprises a first rotating dial and a first cable, the first cable is coupled to the first support and the third support, the second tightening mechanism comprises a second rotating dial and a second cable, the second cable is coupled to the second support and the fourth support.

20. A method for providing stability to a knee joint, comprising:
providing the knee brace in claim 17;
positioning the distal portion of the flexible textile substrate around the calf of the wearer and below the knee of the wearer;
positioning the proximal portion of the flexible textile substrate around the thigh of the wearer and above the knee of the wearer;
positioning the proximal portion of the flexible textile substrate and the distal portion of the flexible textile substrate around the knee of the wearer;
positioning the first support and the second support on a lateral side of the wearer;
positioning the third support and the fourth support on a medial side of the wearer;
tightening a first tightening mechanism to incrementally tension the distal portion of the flexible textile substrate by rotating a first rotating dial to increase tension in a first cable;
tightening a second tightening mechanism to incrementally tension the proximal portion by rotating a second rotating dial to increase tension in a second cable;
loosening the first tightening mechanism to release tension in the distal portion by releasing tension in the first cable; and
loosening the second tightening mechanism to release tension in the proximal portion by releasing tension in the second cable.
